(19) United States
(12) Patent Application Publication
(10) Pub. No.: US 2015/0320387 A1
(43) Pub. Date: Nov. 12, 2015

(54) MEDICAL ACOUSTIC COUPLER

(71) Applicants: KABUSHIKI KAISHA TOSHIBA, Tokyo (JP); TOSHIBA MEDICAL SYSTEMS CORPORATION, Tochigi (JP)

(72) Inventors: Takashi KUBOTA, Tochigi (JP); Takashi TAKEUCHI, Tochigi (JP); Koichi SHIBAMOTO, Tochigi (JP); Satoru TEZUKA, Tochigi (JP)

(73) Assignees: KABUSHIKI KAISHA TOSHIBA, Tokyo (JP); TOSHIBA MEDICAL SYSTEMS CORPORATION, Tochigi (JP)

(21) Appl. No.: 14/806,445

(22) Filed: Jul. 22, 2015

Related U.S. Application Data
(63) Continuation of application No. PCT/JP2014/052626, filed on Feb. 5, 2014.

(30) Foreign Application Priority Data
Feb. 28, 2013 (JP) 2013-039014
Feb. 4, 2014 (JP) 2014-019482

(51) Int. Cl. A61B 8/00

(52) U.S. Cl. CPC A61B 8/4281

(57) ABSTRACT

According to one embodiment, a medical acoustic coupler detachably is provided on an ultrasonic wave radiation surface of an ultrasonic probe. The medical acoustic coupler includes an elastic body. The elastic body has an adhesive force. The elastic body adheres to the ultrasonic wave radiation surface with the adhesive force. The elastic body includes a middle portion, end portions, and groove portions. The middle portion has a size corresponding to the ultrasonic wave radiation surface. The end portions adheres to two opposite side surfaces of the ultrasonic probe. The groove portions are provided between each of the end portions and the middle portion. The groove portions are thinner than the middle portion and the end portions.

### CROSS-REFERENCE TO RELATED APPLICATIONS

This application is a Continuation Application of PCT Application No.PCT/JP2014/052626, filed Feb. 5, 2014 and based upon and claims the benefit of priority from the Japanese Patent Application No. 2013-039014, filed Feb. 28, 2013 and the Japanese Patent Application No. 2014-019482, filed Feb. 4, 2014, the entire contents of all of which are incorporated herein by reference.

### FIELD

Embodiments described herein relate generally to a medical acoustic coupler.

### BACKGROUND

Conventionally, when an ultrasonic probe is made to abut against an object, an acoustic coupler is sometimes arranged between the ultrasonic probe and the object. The acoustic coupler has an acoustic impedance between an acoustic impedance near an object surface and the acoustic impedance of the acoustic radiation surface of the ultrasonic probe. The acoustic coupler is formed from a plate-like elastic body (an elastic gel or the like). The acoustic coupler contributes to an improvement in the quality of an ultrasonic image corresponding to a portion near the body surface of an object. In addition, the acoustic coupler can ensure acoustic coupling between an object surface having irregularity and the ultrasonic wave radiation surface of the ultrasonic probe.

The acoustic coupler has flexibility and adhesiveness. With these properties, the acoustic coupler can adhere to the ultrasonic probe along its outer shape. In order to ensure the shape retention and operability of the acoustic coupler, a thin film having low adhesiveness is sometimes provided on the surface side of the acoustic coupler to which the ultrasonic probe adheres.

For example, owing to an increase in the thickness of the acoustic coupler, an increase in the rigidity of the acoustic coupler, insufficient adhesiveness, or the like, the elastic force of the acoustic coupler itself, which adheres to the ultrasonic probe while being bent along its outer shape, makes it easy to peel off the ultrasonic probe.

### BRIEF DESCRIPTION OF THE DRAWINGS

FIG. 1 is a view showing the outer shape of the abutment surface of a medical acoustic coupler according to the first embodiment.

FIG. 2 is a view showing an example of groove portions each having a V-shaped cross-sectional shape on a cross-section of FIG. 1 taken along a-a′ according to the first embodiment.

FIG. 3 is a view showing a state in which the medical acoustic coupler from which a protective film is peeled off adheres to the ultrasonic wave radiation surface of an ultrasonic probe according to the first embodiment.

FIG. 4 is view showing an example of how the medical acoustic coupler is bent along the outer shape of the ultrasonic probe to make the entire area of the medical acoustic coupler adhere to the ultrasonic probe according to the first embodiment.

FIG. 5 is a view showing an example of how the entire area of the medical acoustic coupler is made to adhere to the ultrasonic probe according to the first embodiment.

FIG. 6 is a view showing an example of groove portions each having a U-shaped cross-sectional shape on a cross-section of FIG. 1 taken along a-a′ according to a modification of the first embodiment.

FIG. 7 is a view showing an example of groove portions each having a semicircular cross-sectional shape on a cross-section of FIG. 1 taken along a-a′ according to a modification of the first embodiment.

FIG. 8 is a view showing an example of groove portions each having a concave cross-sectional shape on a cross-section of FIG. 1 taken along a-a′ according to a modification of the first embodiment.

FIG. 9 is a view showing the outer shape of the abutment surface of a medical acoustic coupler according to the second embodiment.

FIG. 10 is a cross-sectional view of FIG. 9 taken along a-a′ according to the second embodiment.

FIG. 11 is a view showing a state in which the medical acoustic coupler from which a protective film is peeled off adheres to the ultrasonic wave radiation surface of an ultrasonic probe according to the second embodiment.

FIG. 12 is view showing an example of how the medical acoustic coupler is bent along the outer shape of the ultrasonic probe to make the entire area of the medical acoustic coupler adhere to the ultrasonic probe according to the second embodiment.

### DETAILED DESCRIPTION

In general, according to one embodiment, a medical acoustic coupler detachably is provided on an ultrasonic wave radiation surface of an ultrasonic probe. The medical acoustic coupler includes an elastic body. The elastic body has an adhesive force. The elastic body adheres to the ultrasonic wave radiation surface with the adhesive force. The elastic body includes a middle portion, end portions, and groove portions.

The middle portion has a size corresponding to the ultrasonic wave radiation surface. The end portions adheres to two opposite side surfaces of the ultrasonic probe. The groove portions are provided between each of the end portions and the middle portion. The groove portions are thinner than the middle portion and the end portions.

The embodiments will be described below with reference to the accompanying drawings. Note that the same reference numerals in the following description denote constituent elements having almost the same arrangements, and a repetitive description will be made only when required.

### First Embodiment

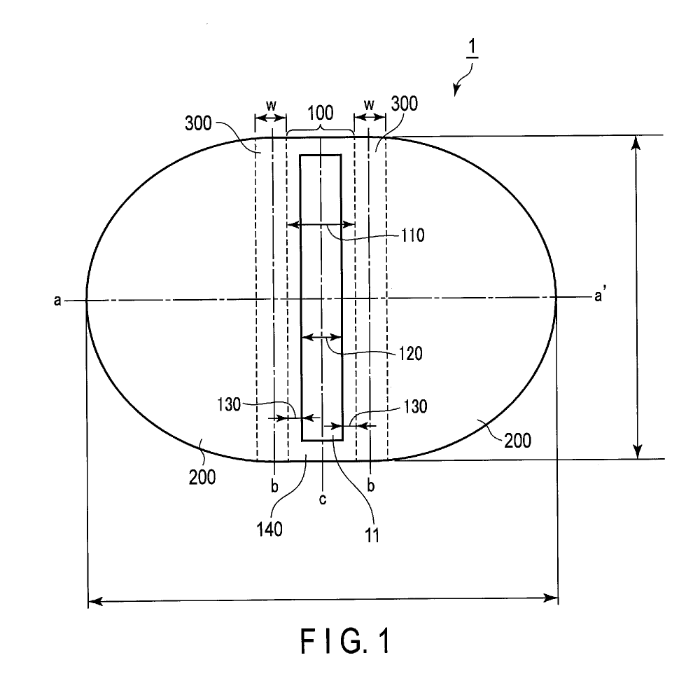

FIG. 1 is a view showing the outer shape of a surface (to be referred to as an abutment surface hereinafter) of a medical acoustic coupler 1 according to the first embodiment which abuts against an object. As shown in

FIG. 1, the medical acoustic coupler 1 has an almost elliptic outer shape. Note that the outer shape of the medical acoustic coupler 1 is not limited to an almost elliptic shape and may be a rectangular shape depending on the shape of an ultrasonic probe to which the coupler is to adhere. The medical acoustic coupler 1 is detachably provided on the ultrasonic wave radiation surface of the ultrasonic probe.

The medical acoustic coupler 1 is a flexible composite elastic body. This allows the medical acoustic coupler 1 to deform along the outer shape of the ultrasonic probe. More specifically, the medical acoustic coupler 1 includes an elastic gel-like layer having a predetermined thickness and a film layer provided on a surface including an abutment surface which abuts against an object. The film layer is provided on the opposite surface of the gel-like layer which faces the adhesive surface to which the ultrasonic probe is to adhere.

The gel-like layer is, for example, a gel-like elastic body made of polyurethane as a main component. The gel-like elastic body is, for example, a polyurethane gel. A polyurethane gel has predetermined flexibility or hardness that can conform to the outer shape of the ultrasonic probe, mechanical strength that can endure frequent attachment/detachment to/from the ultrasonic probe, safety for an object, and water resistance in addition to adhesiveness (to be described later).

The gel-like layer has an acoustic impedance of, for example, 1.1 MRayl to 2 MRayl. This acoustic impedance range contributes to an improvement in acoustic coupling between the acoustic radiation surface of the ultrasonic probe and an object surface against which the ultrasonic probe abuts through the medical acoustic coupler.

In addition, the gel-like layer is, for example, an elastic body having a predetermined adhesive force. The predetermined adhesive force is that which can support a weight equal to or more than that of the medical acoustic coupler at the entire surface of the gel-like layer. In addition, the predetermined adhesive force is that which allows the operator to peel off the medical acoustic coupler 1 adhering to the ultrasonic probe. With the predetermined adhesive force, for example, the medical acoustic coupler 1 adheres to and is held on the ultrasonic probe. In addition, the medical acoustic coupler 1 adhering to the ultrasonic probe can be peeled off the ultrasonic probe by the operator. The gel-like layer has a thickness of 1 mm to 10 mm.

This thickness range allows the medical acoustic coupler 1 to deform along the outer shape of the ultrasonic probe and attach it to the ultrasonic probe in tight contact with each other. In addition, the thickness range allows the medical acoustic coupler 1 to contribute to the generation of a good ultrasonic image of a living tissue near the body surface of an object. In addition, the medical acoustic coupler 1 having the thickness range can improve follow-up property with respect to the ultrasonic probe as compared with a medical acoustic coupler falling outside the thickness range. In addition, if the thickness of the gel-like layer falls within the range of 1 mm to 5 mm, a better ultrasonic image can be obtained, and follow-up property with respect to the ultrasonic probe can be improved.

The film layer has higher rigidity than the gel-like layer. The film layer is a uniform layer made of, as a main component, one of for example, polypropylene, polyester, polyethylene, polyethylene terephthalate, polyimide, polyamide, polyvinylidene chloride, polyvinyl chloride, polymethylpentene, polyolefin, and polyphenylenesulfide. More specifically, the film layer has a Young's modulus of, for example, 0.2 GPa to 5 GPa. This rigidity allow the film layer to improve the rigidity of the medical acoustic coupler 1. More specifically, the film layer can prevent severance, breakage, or the like of the gel-like layer.

The film layer has an acoustic impedance of, for example, 1.6 MRayl to 5 MRayl. This acoustic impedance range contributes to an improvement in acoustic coupling between the acoustic radiation surface of the ultrasonic probe and the gel-like layer. The film layer has a lower adhesive force than the gel-like layer. More specifically, the adhesive force of the surface of the film layer is less than that corresponding to the weight of the medical acoustic coupler 1. With this adhesive force of the film layer, the operator can peel off the ultrasonic probe attached to the medical acoustic coupler 1 from the object body surface against which the coupler abuts.

The film layer has a thickness equal to or less than 1/20 that of the gel-like layer. More specifically, the thickness of the film layer falls within the range of, for example, 0.005 mm to 0.1 mm. This thickness range allows the medical acoustic coupler 1 to have adequate flexibility while maintaining proper rigidity. In addition, the film layer can properly keep acoustic coupling between the acoustic radiation surface of the ultrasonic probe and the gel-like layer.

The surface of the gel-like layer which faces the film layer through the gel-like layer is provided with a protective sheet for protecting the gel-like layer before the gel-like layer is made to adhere to the ultrasonic probe. The protective sheet is removed when the medical acoustic coupler 1 is made to adhere to the ultrasonic probe.

As shown in FIG. 1, the gel-like layer of the medical acoustic coupler 1 includes a middle portion 100 (flat portion) having a size (e.g., an area or shape) corresponding to a region 11 corresponding to the ultrasonic wave radiation surface of the ultrasonic probe, a plurality of end portions 200 to be made to adhere to the side surfaces of the ultrasonic probe, and groove portions 300 which are provided between the middle portion 100 and the end portions 200 and thinner than the middle portion 100 and the end portions 200. For the sake of easy explanation, assume that the thickness of the middle portion 100 is equal to that of the end portions 200. Note that the thickness of the middle portion 100 may differ from that of the end portions 200.

A width 110 of the middle portion is, for example, 5 mm to 30 mm. The width 110 of the middle portion is determined in accordance with the width of each transducer of the ultrasonic probe. If, for example, the ultrasonic probe is a one-dimensional array ultrasonic probe, the width 110 of the middle portion is the sum of a length 120 of each transducer in the elevation direction and a predetermined width 130 (e.g., a region corresponding to a footprint 140).

As shown in FIG. 1, the groove portions 300 are provided parallel to the two opposite long sides of the middle portion 100 in a rectangular shape. Note that the groove portions 300 may be provided parallel to the two opposite short sides of the middle portion 100. In addition, if the middle portion 100 has a square shape, the groove portions 300 may be provided parallel to two arbitrary opposite sides of the square. Furthermore, if the middle portion 100 has a circular shape (corresponds to an annular array probe), the groove portion 300 is provided around the middle portion 100 so as to enclose it.

The groove portions 300 has a thickness smaller than that of the gel-like layer (the thickness of the middle portion 100 and the thickness of the end portions 200). More specifically, the thickness of the groove portions 300 is equal to or more than ⅕ and equal to or less than ⅘ the thickness of the middle portion 100. The cross-sectional shape of the groove portions 300 is, for example, a V shape, U shape, semicircular shape, or concave shape. The first embodiment will exemplify the groove portions 300 having a V-shaped cross-sectional shape.

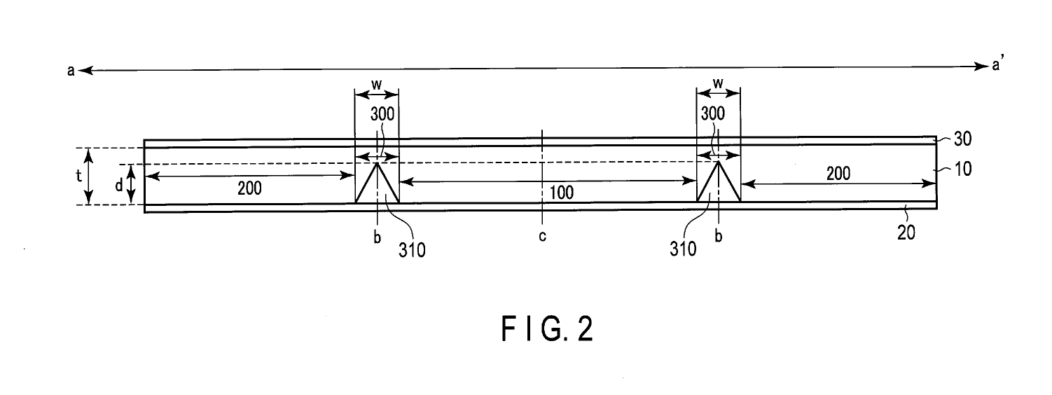

FIG. 2 is a view showing an example of the groove portions 300 each having a V-shaped cross-sectional shape on a cross-section of FIG. 1 taken along a-a′. As shown in FIG. 2, a depth d at a deepest portion b of each groove portion 300 is equal to or more than ⅕ a thickness t of a gel-like layer 10. That is, t≧d≧⅕×t. As shown in FIG. 2, each groove portion 300 has a V-shaped cavity portion 310. A width w of the groove portions 300 is larger than the depth d at the deepest portions of the groove portions 300 (w>d). As shown in FIG. 2, a triangle corresponding to the cavity portion 310 defined by the deepest portion b of each groove portion 300 and the width w of each groove portion 300 is an acute triangle.

The end portions 200 are provided adjacent to the groove portions 300. The end portions 200 from which a protective sheet 20 is peeled off is made to adhere to the side surfaces of the ultrasonic probe. The shape of the end portions 200 conforms to the outer shape of an ultrasonic probe 3.

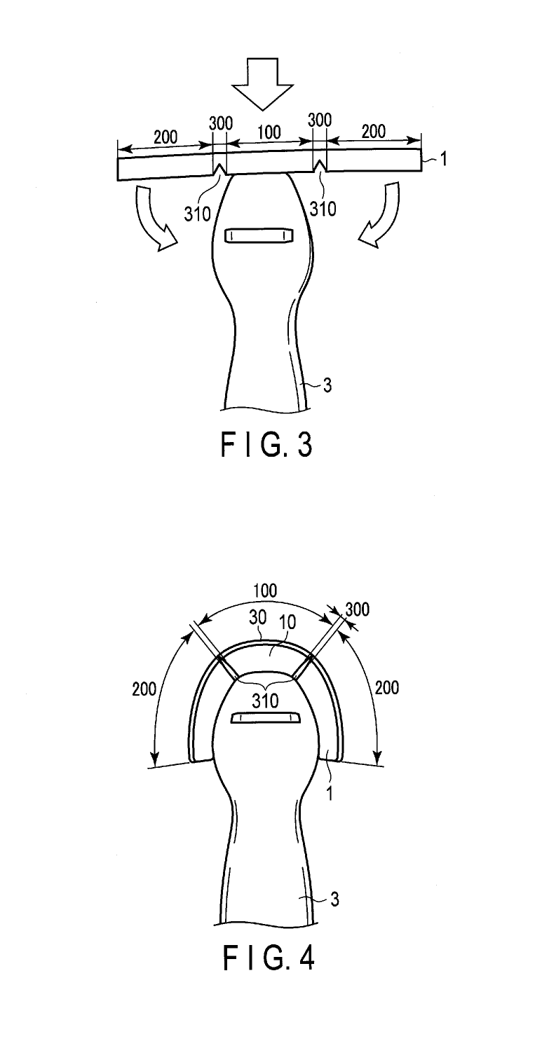

FIG. 3 is a view showing a state in which the medical acoustic coupler 1 from which the protective sheet 20 is peeled off adheres to the ultrasonic wave radiation surface of the ultrasonic probe 3. As shown in FIG. 3, the middle portion 100 of the medical acoustic coupler 1 is made to adhere to the ultrasonic wave radiation surface of the ultrasonic probe 3. At this time, the cavity portions 310 of the groove portions 300 are arranged on the ultrasonic probe 3 side. The arrows in FIG. 3 indicate a method of arranging the medical acoustic coupler 1, from which the protective sheet 20 is peeled off, on the ultrasonic probe 3.

FIG. 4 is view showing an example of how the medical acoustic coupler 1 is bent along the outer shape of the ultrasonic probe 3 to make the entire area of the medical acoustic coupler 1 adhere to the ultrasonic probe 3. As shown in FIG. 4, the end portions 200 are made to adhere to the side surfaces of the ultrasonic probe 3. With this operation, as shown in FIG. 4, the cavity portions 310 of the groove portions 300 of the medical acoustic coupler 1 are closed tightly by the gel-like layer 10.

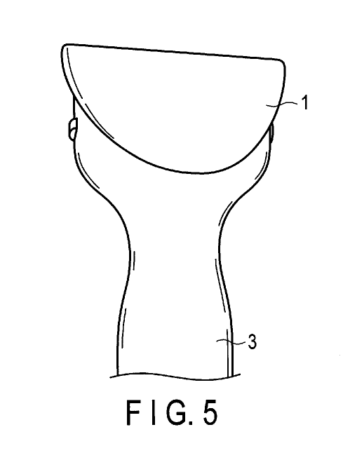

FIG. 5 is a view showing an example of how the entire area of the medical acoustic coupler 1 is made to adhere to the ultrasonic probe 3. FIG. 5 is a view viewed from a direction perpendicular to the visual line direction in FIG. 4. As shown in FIG. 5, the end portions 200 of the medical acoustic coupler 1 are made to adhere to the side surfaces of the ultrasonic probe 3.

### (Modification)

A difference from the first embodiment is that the groove portions 300 each have a cross-sectional shape different from a V shape.

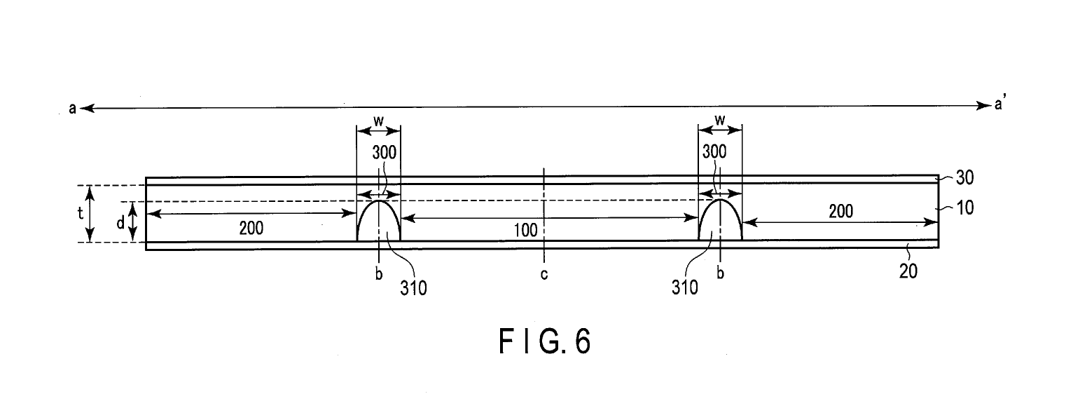

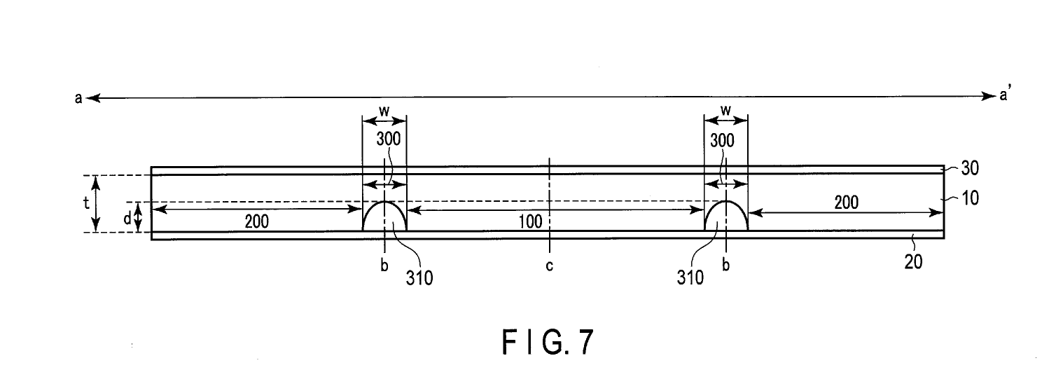

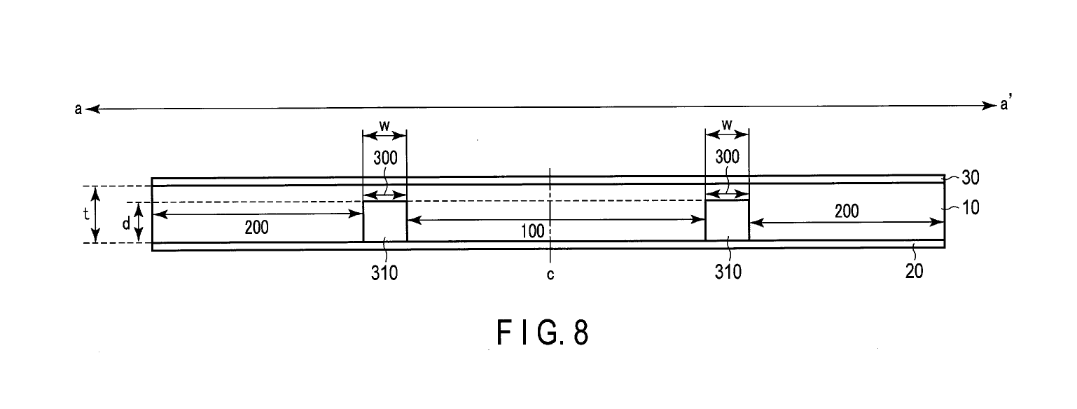

FIG. 6 is a view showing an example of the groove portions 300 each having a U-shaped cross-sectional shape on a cross-section of FIG. 1 taken along a-a′. FIG. 7 is a view showing an example of the groove portions 300 having a semicircular cross-sectional shape on a cross-section of FIG. 1 taken along a-a′. As shown in FIGS. 6 and 7, the position of the deepest portion b of each groove portion 300 on the protective sheet 20 is located within the width w of the groove portion 300. FIG. 8 is a view showing an example of the groove portions 300 having a concave cross-sectional shape on a cross-section of FIG. 1 taken along a-a′. As shown in FIG. 8, the width of the deepest portion of each groove portion 300 is equal to the width w of the groove portion 300.

According to the arrangement described above, the following effects can be obtained.

The medical acoustic coupler 1 according to the first embodiment has, in the gel-like layer 10, the groove portions 300 having the cavity portions 310 between the middle portion 100 and the plurality of end portions 200. The medical acoustic coupler 1 can be made to adhere to the ultrasonic probe 3 in tight contact with each other by being deformed along the outer shape of the ultrasonic probe 3. That is, making the cavity portions 310 of the groove portions 300 close tightly can reduce the elastic force of the deformed gel-like layer 10. As described above, according to the medical acoustic coupler 1, it is possible to reduce the peeling of the medical acoustic coupler 1 from the ultrasonic probe 3.

In addition, the medical acoustic coupler 1 can compensate for a reduction in the rigidity of the gel-like layer 10 at the groove portions 300 by using a film layer 30. This allows the medical acoustic coupler 1 to have the groove portions 300 which are formed to have a depth and a width in accordance with the angles defined by the ultrasonic wave radiation surface of the ultrasonic probe 3 and the side surfaces and the thickness of the gel-like layer 10. In addition, each groove portion 300 can be formed into a shape corresponding to a bending angle.

Furthermore, according to the medical acoustic coupler 1, the groove portions 300 can improve the adhesion between the medical acoustic coupler 1 and the ultrasonic probe 3. That is, when the medical acoustic coupler 1 is attached to the ultrasonic probe 3, since no crease occurs in the medical acoustic coupler 1, air does not easily enter between the medical acoustic coupler 1 and the ultrasonic probe 3. This improves the acoustic matching between the ultrasonic probe 3 and the abutment surface of an object. As described above, using the medical acoustic coupler 1 can improve the quality of an ultrasonic image.

As described above, the medical acoustic coupler 1 according to the first embodiment can solve the problem that, owing to an increase in the thickness of the medical acoustic coupler 1, an increase in the rigidity of the medical acoustic coupler 1, insufficient adhesiveness, or the like, the elastic force of the medical acoustic coupler 1 itself makes it easy to peel off the ultrasonic probe 3.

### Second Embodiment

A difference from the first embodiment is that a medical acoustic coupler 1 includes an elastic body having a size corresponding to an ultrasonic wave radiation surface and a film layer which is provided on the elastic body, has an adhesive force that can support a weight equal to or more than the weight of the medical acoustic coupler 1, and can adhere to the two opposite side surfaces of an ultrasonic probe 3.

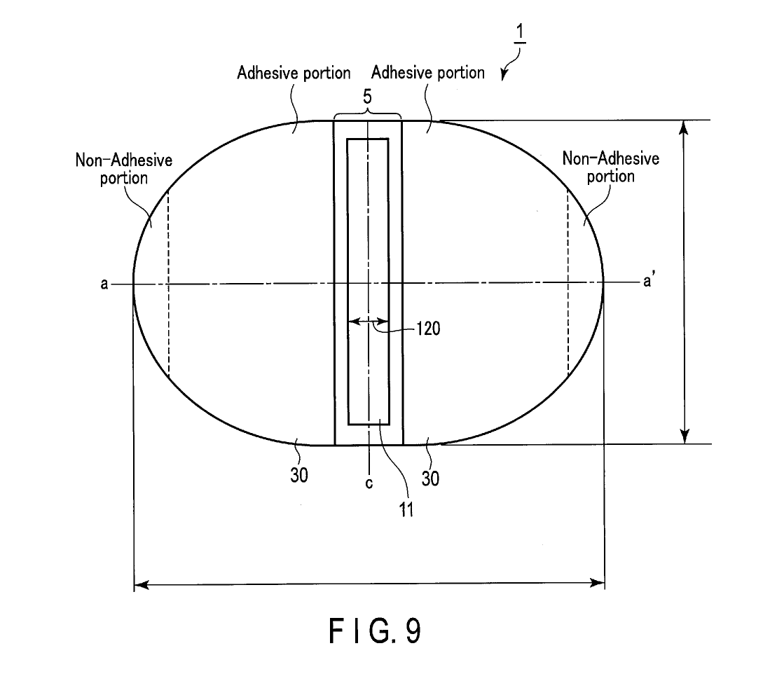

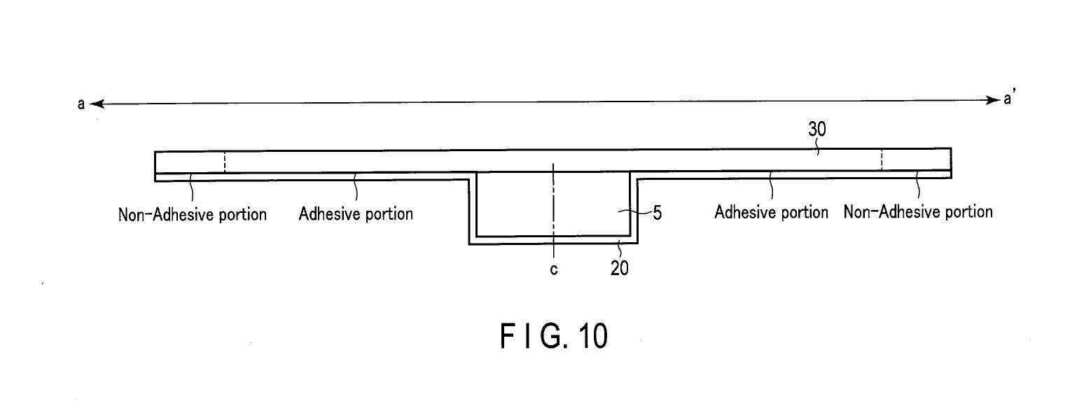

FIG. 9 is a view showing the outer shape of the abutment surface of the medical acoustic coupler 1 according to the second embodiment which abuts against an object. FIG. 10 is a cross-sectional view of FIG. 9 taken along a-a′. As shown in FIGS. 9 and 10, the medical acoustic coupler 1 includes an elastic body 5 and a film layer 30.

The elastic body 5 is, for example, a gel-like layer serving as an acoustic matching layer. The elastic body 5 has an adhesive surface which can adhere to the ultrasonic wave radiation surface of the ultrasonic probe 3. The elastic body 5 has a size corresponding to the ultrasonic wave radiation surface of the ultrasonic probe 3. That is, the adhesive surface of the elastic body 5 is wide enough to include the region 11 corresponding to the ultrasonic wave radiation surface, as shown in, for example, FIG. 9. If the ultrasonic probe 3 is a one-dimensional array ultrasonic probe 3, the elastic body 5 has a width larger than a length 120 of each transducer in the elevation direction. As shown in FIG. 10, a protective sheet 20 is provided on the adhesive surface and side surfaces of the elastic body 5 and the lower surface of the film layer 30 before the medical acoustic coupler 1 is attached to the ultrasonic probe.

The film layer 30 is provided on the opposite surface of the elastic body 5 which faces the adhesive surface. The film layer 30 is thinner than the elastic body 5. The film layer 30 has adhesive portions and non-adhesive portions. The adhesive portions of the film layer 30 are made to adhere to the two side surfaces of the ultrasonic probe 3 which face each other. The adhesive portions of the film layer 30 each have an adhesive force that can support a weight equal to or more than, for example, the weight of the medical acoustic coupler 1. As shown in

FIGS. 9 and 10, the non-adhesive portions are provided on the end portions of the film layer 30. Note that the non-adhesive portions may be omitted. That is, the lower surface of the film layer 30 may be formed from an adhesive portion.

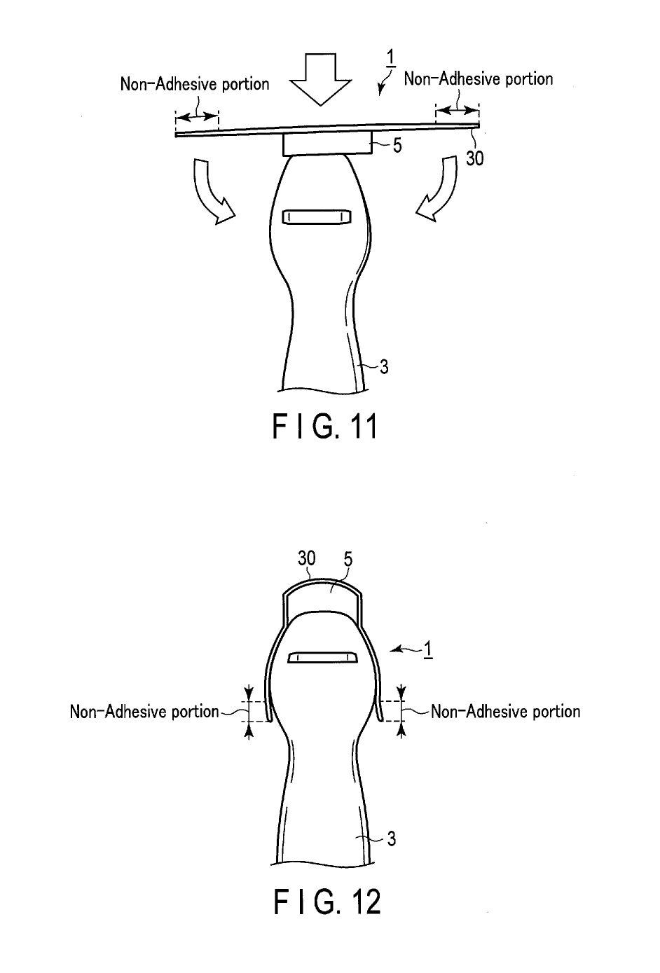

When the medical acoustic coupler 1 is attached to the ultrasonic probe, the protective sheet 20 is removed from the elastic body 5, the adhesive surface is made to adhere to the ultrasonic wave radiation surface. FIG. 11 is a view showing a state in which the medical acoustic coupler 1 from which the protective film 20 is peeled off adheres to the ultrasonic wave radiation surface of the ultrasonic probe 3. As shown in FIG. 11, the elastic body 5 of the medical acoustic coupler 1 is made to adhere to the ultrasonic wave radiation surface of the ultrasonic probe 3. The arrows in FIG. 11 indicate an example of an arrangement method of arranging the medical acoustic coupler 1, from which the protective sheet 20 is peeled off, on the ultrasonic probe 3.

FIG. 12 is a view showing an example of how the medical acoustic coupler 1 is bent along the outer shape of the ultrasonic probe 3 to make the entire area of the medical acoustic coupler 1 adhere to the ultrasonic probe 3. As shown in FIG. 12, the adhesive portions of the film layer 30 are made to adhere to the two side surfaces of the ultrasonic probe 3. As shown in FIG. 12, this makes the elastic body 5 of the medical acoustic coupler 1 and the film layer 30 adhere to the ultrasonic probe 3. It is possible to remove the medical acoustic coupler 1 from the ultrasonic probe 3 by using the non-adhesive portions of the film layer 30.

According to the above arrangement, the following effects can be obtained.

The medical acoustic coupler 1 according to the second embodiment includes the elastic body 5 having the adhesive surface which has a size corresponding to the ultrasonic wave radiation surface and can adhere to the ultrasonic wave radiation surface and the film layer which is provided on an opposite surface facing the adhesive surface and is made to adhere to the two side surfaces of the ultrasonic probe 3. The medical acoustic coupler 1 can be made to adhere to the ultrasonic probe 3 in tight contact by being deformed along the outer shape of the ultrasonic probe 3. As described above, according to the medical acoustic coupler 1, it is possible to reduce the peeling of the medical acoustic coupler 1 from the ultrasonic probe 3.

In addition, according to the medical acoustic coupler 1, the elastic body 5 having a size corresponding to the ultrasonic wave radiation surface can improve the adhesion between the medical acoustic coupler 1 and the ultrasonic probe 3. That is, when the medical acoustic coupler 1 is attached to the ultrasonic probe 3, since no crease occurs in the medical acoustic coupler 1, air does not easily enter between the medical acoustic coupler 1 and the ultrasonic probe 3. This improves the acoustic matching between the ultrasonic probe 3 and the abutment surface of an object. As described above, using the medical acoustic coupler 1 can improve the quality of an ultrasonic image. In addition, the operator can more easily remove the medical acoustic coupler 1 from the ultrasonic probe 3 by holding the non-adhesive portions of the film layer 30.

While certain embodiments have been described, these embodiments have been presented by way of example only, and are not intended to limit the scope of the inventions.

Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. A medical acoustic coupler detachably provided on an ultrasonic wave radiation surface of an ultrasonic probe, comprising an elastic body having an adhesive force with which the elastic body is configured to adhere to the ultrasonic wave radiation surface,
the elastic body including
a middle portion having a size corresponding to the ultrasonic wave radiation surface,
a plurality of end portions configured to adhere to two opposite side surfaces of the ultrasonic probe, and
groove portions which are provided between each of the end portions and the middle portion and are thinner than the middle portion and the end portions.

2. The medical acoustic coupler according to claim 1, wherein the middle portion has a rectangular shape, and
the groove portions are provided parallel to two opposite sides of the rectangular shape.

3. The medical acoustic coupler according to claim 1, further comprising a film layer provided on an opposite surface of the elastic body which faces an adhesive surface made to adhere to the ultrasonic probe,
the film layer having a larger thickness than the middle portion, a smaller adhesive force than the adhesive force, and higher rigidity than the elastic body.

4. The medical acoustic coupler according to claim 1, wherein a depth of the groove portion is not less than ⅕ a thickness of the middle portion.

5. The medical acoustic coupler according to claim 1, wherein a cross-sectional shape of the groove portion is one of a concave shape, a V shape, a U shape, and a semicircular shape.

6. The medical acoustic coupler according to claim 1, wherein a width of the groove portion is larger than a thickness of the middle portion.

7. The medical acoustic coupler according to claim 3, wherein an acoustic impedance of the elastic body is 1.1 MRayl to 2 MRayl,
an acoustic impedance of the film layer is 1.6 MRayl to 4 MRayl,
a Young's modulus of the film layer is 0.2 GPa to 5 GPa, and
a thickness of the film layer is not more than 1/20 a thickness of the elastic body.

8. A medical acoustic coupler detachably provided on an ultrasonic wave radiation surface of an ultrasonic probe, comprising:
an elastic body having a size corresponding to the ultrasonic wave radiation surface and an adhesive surface configured to adhere to the ultrasonic wave radiation surface; and
a film layer provided on an opposite surface facing the adhesive surface, having a smaller thickness than the elastic body, and configured to adhere to two opposite side surfaces of the ultrasonic probe.

9. The medical acoustic coupler according to claim 8, wherein an end portion of the film layer includes a non-adhesive portion.

10. The medical acoustic coupler according to claim 8, wherein an acoustic impedance of the elastic body is 1.1 MRayl to 2 MRayl,
an acoustic impedance of the film layer is 1.6 MRayl to 4 MRayl,
a Young's modulus of the film layer is 0.2 GPa to 5 GPa, and
a thickness of the film layer is not more than 1/20 a thickness of the elastic body.
